(19) United States
(12) Patent Application Publication
(10) Pub. No.: US 2008/0268024 A1
(43) Pub. Date: Oct. 30, 2008

(54) Compositions for Oral and/or Topical Administration

(76) Inventors: Santiago Rull Prous, Barcelona (ES); Bernd Fabry, Korschenbroich (DE); Doris Bell, Duesseldorg (DE)

Correspondence Address: SYNNESTVEDT & LECHNER LLP, 1101 MARKET STREET, PHILADELPHIA, PA, 19107, US

(21) Appl. No.: 11/569,615

(22) PCT Filed: May 14, 2005
(86) PCT No.: PCT/EP05/05316

§ 371 Date: Feb. 28, 2008

(30) Foreign Application Priority Data
May 25, 2004 (EP) 04012288.9

(51) Int. Cl. A61K 47/10; A61P 1/00

(52) U.S. Cl. 424/439; 424/780; 514/772; 514/54; 514/61; 514/53; 514/58; 424/93.1

(57) ABSTRACT

Compositions for oral and/or topical administration of a prebiotic and a polyphenol or a plant extract containing a polyphenol, and mixtures thereof, are disclosed. The compositions are disclosed as enhancing the body's population of beneficial microorganisms for improving health and well-being.

### CROSS REFERENCE TO RELATED APPLICATIONS

This national phase application is filed under 35 U.S.C. § 371 from International Application No. PCT/EP2005/005316 filed May 14, 2005, which designated the United States of America and which claims priority from European application EP 04012288.9 filed May 25, 2004; the entire contents of each application are hereby incorporated herein by reference.

### FIELD OF THE INVENTION

The present invention is related to the area of alimentation and concerns oral and/or topical compositions comprising defined active principles and prebiotics, dietary supplements and food compositions comprising said actives and prebiotics, and the use of mixtures comprising said actives and prebiotics for improving the stimulation of the growth of healthy bacteria.

### BACKGROUND OF THE INVENTION

Probiotics contain live bacteria and represent an important part of the complex world of foods that are good for health. It is the bacteria and the metabolites which they produce that give these products their health promoting properties. The best known example of a probiotic is yogurt. The experimental data for yogurt is still not as conclusive as one would like, however, human studies related to the consumption of dietary milk products show increased milk digestibility, quicker recovery from certain types of diarrhea, enhanced immune function, relation in certain cancers, and possible lowering of blood cholesterol levels.

Bacteria found in products like yogurt, kefir or fermented vegetables usually aren't found in the human intestine. In fact, the intestinal environment is often a hostile one for these foreign bacteria. Because of this, bacteria eaten in probiotic products don't colonise the intestine but are flushed through and eliminated from the body.

The bacteria living in the intestine make up a very large and very diverse population. The numbers of each kind of bacteria change depending on age, diet, health status, and use of drugs and supplements. The effects are linked to the ability of the bacteria to adhere to the intestinal wall and use the semi-digested food that is passing through the intestines. It is not surprising to have found that the bacterial population in the intestines of vegetarians is much different compared to that of meat eaters. Because some bacteria have specific nutrient requirements, it has been proposed that adding these particular foods or nutrients to the diet could be a way of increasing the numbers of specific bacteria. Such additives are called “prebiotics”. Thus, to be effective, prebiotics must escape digestion in the upper gastrointestinal tract and be used by a limited number of the microorganisms comprising the colonic microflora. In the large intestine, prebiotics are converted into short-chain fatty acids like capronic or caprylic acid. Said acids are used by the human body as an energy source. Besides this, the short-chain acids are known to inhibit inflammation of the intestine, which represents a kind of cancer prophylaxis. In addition, prebiotics increase the resorption time in the intestine which leads to an improved uptake of minerals. Typical examples for well-known prebiotics are oligosaccharides, e.g. in 1995, Gibson et al. found that oligofructose and inulin, when fed to humans, selectively stimulated the growth of bifidobacteria without influencing the numbers of lactobacillus. Since prebiotics mainly stimulate the growth of bifidobacteria, they also are referred to as bifidogenetic factors.

Although various types of prebiotics are known from the literature and can be found in the market, there is still an increasing need for more active alternatives or additives which support the various activities of existing products in a synergistic manner. Therefore, the object of the present invention has been to provide a new system of prebiotic compounds, which shows a synergistic stimulation of the growth of healthy bacteria, preferably bifido and lactic bacteria both, and improves the health status of the human body.

### DETAILED DESCRIPTION OF THE INVENTION

The terms “prebiotics”, “polyphenols” and “plant extracts” used below and throughout the specification shall be understood to encompass one or more.

The present invention provides oral and/or topical compositions, comprising
(a) prebiotics; and
(b) polyphenols or plant extracts rich in polyphenols, and mixtures thereof.

Surprisingly, it has been observed that mixtures of various types of polyphenols preferably of plant origin and prebiotics show a synergistic behavior with respect to stimulation of growth of bacteria selected from the group consisting of Bifidobacterium breve, Bifidobacterium infantis, Bifidobacterium longum and Bifidobacterium adolescentis on one hand, and Lactobacillus bulgaricus, Lactobacillus acidophilus, Lactobacillus casei, Lactobacillus plantarum, Streptococcus faecium, and Streptococcus thermophilus on the other.

### Prebiotics

Prebiotics are defined as non-digestible food ingredients that may beneficially affect the host by selectively stimulating the growth and/or the activity of a limited number of bacteria in the colon. The following describes the various oligosaccharides which can be taken into account as suitable prebiotics (component a):

Fructooligosaccharides
Fructooligosaccharides or FOS typically refer to short-chain oligosaccharides comprised of D-fructose and D-glucose, containing from three to five monosaccharide units. FOS, also called neosugar and short-chain FOS, are produced on a commercial scale from sucrose using a fungal fructosyltransferase enzyme. FOS are resistant to digestion in the upper gastrointestinal tract. They act to stimulate the growth of Bifidobacterium species in the large intestine. FOS are marketed in the United States in combination with probiotic bacteria and in some functional food products.

Inulins
Inulins refer to a group of naturally-occurring fructose-containing oligosaccharides. Inulins belong to a class of carbohydrates known as fructans. They are derived from the roots of chicory (Cichorium intybus) and Jerusalem artichokes. Inulins are mainly comprised of fructose units and typically have a terminal glucose. The bond between fructose units in inulins is a beta-(2-1) glycosidic linkage. The average degree of polymerisation of inulins marketed as nutritional supplements is 10 to 12. Inulins stimulate the growth of Bifidobacterium species in the large intestine.

Isomaltooligosaccharides
Isomaltooligosaccharides comprise a mixture of alpha-D-linked glucose oligomers, including isomaltose, panose, isomaltotetraose, isomaltopentaose, nigerose, kojibiose, isopanose and higher branched oligosaccharides. Isomaltooligosaccharides are produced by various enzymatic processes. They act to stimulate the growth of Bifidobacterium species and Lactobacillus species in the large intestine. Isomaltooligosaccharides are marketed in Japan as dietary supplements and in functional foods. They are being developed in the United States for similar uses.

Lactilol
Lactilol is a disaccharide analogue of lactulose. Its pharmaceutical use is in the treatment of constipation and hepatic encephalopathy. Lactilol is also used in Japan as a prebiotic. It is resistant to digestion in the upper gastrointestinal tract and is fermented by a limited number of colonic bacteria, resulting in an increase in the biomass of bifidobacteria and lactobacilli in the colon. Lactilol is known chemically as 4-O-(beta-D-galactopyranosyl)-D-glucitol. Lactilol is not approved for the treatment of hepatic encephalopathy or constipation in the U.S., and its use as a prebiotic is considered experimental. Lactilol is used in Europe as a food sweetener.

Lactosucrose
Lactosucrose is a trisaccharide comprised of D-galactose, D-glucose and D-fructose. Lactosucrose is produced enzymatically by the enzymatic transfer of the galactosyl residue in lactose to sucrose. Lactosucrose is resistant to digestion in the stomach and small intestine. It is selectively utilized by intestinal Bifidobacterium species resulting in significant induction of growth of these bacteria in the colon. Therefore, under physiological conditions, lactosucrose acts on the intestinal microflora as a growth factor for Bifidobacterium species. Lactosucrose is also known as 4G-beta-D-galactosylsucrose. It is widely used in Japan as a dietary supplement and in functional foods, including yogurt. Lactosucrose is being developed in the United States for similar uses.

Lactulose
Lactulose is a semi-synthetic disaccharide comprised of the sugars D-lactose and D-fructose. The sugars are joined by a beta-glycosidic linkage, making it resistant to hydrolysis by human digestive enzymes. Lactulose is, however, fermented by a limited number of colonic bacteria. This can lead to changes in the colonic ecosystem in favour of bacteria, such as lactobacilli and bifidobacteria, which may confer some health benefits. Lactulose is a prescription drug in the United States for the treatment of constipation and hepatic encephalopathy. It is marketed in Japan for use as a dietary supplement and in functional foods. Its use in the United States as a prebiotic substance is still experimental.

Pyrodextrins
Pyrodextrins comprise a mixture of glucose-containing oligosaccharides that is derived from the hydrolysis of starch. Pyrodextrins have been found to promote the proliferation of Bifidobacterium species in the large intestine. They are resistant to digestion in the upper gastrointestinal tract. Pyrodextrins are being developed for the nutritional supplement market place.

Soy Oligosaccharides
Soy oligosaccharides refer to oligosaccharides found in soybeans and also in other beans and peas. The two principal soy oligosaccharides are the trisaccharide raffinose and the tetrasaccharide stachyose. Raffinose comprises one molecule each of D-galactose, D-glucose and D-fructose. Stachyose consists of two molecules of D-galactose, one molecule of D-glucose and one molecule of D-fructose. Soy oligosaccharides act to stimulate the growth of Bifidobacterium species in the large intestine. They are marketed in Japan as dietary supplements and in functional foods. They are being developed in the United States for similar uses.

Transgalactooligosaccharides
Transgalactooligosaccharides (TOS) are a mixture of oligosaccharides consisting of D-glucose and D-galactose. TOS are produced from D-lactose via the action of the enzyme beta-galactosidase obtained from Aspergillus oryzae. TOS are resistant to digestion in the upper gastrointestinal tract and stimulate the growth of bifidobacteria in the large intestine. TOS are marketed in Japan and Europe as dietary supplements and are used in functional foods. They are being developed for similar use in the United States.

Xylooligosaccharides
Xylooligosaccharides are comprised of oligosaccharides containing beta (1→4) linked xylose residues. The degree of polymerisation of xylooligosaccharides is from two to four. Xylooligosaccharides are obtained by enzymatic hydrolysis of the polysaccharide xylan. They are marketed in Japan as prebiotics and are being developed for similar use in the United States.

Biopolymers
Suitable biopolymers like e.g. beta-glucans include those originating from plants including cereals such as oats and barley, fungi, yeast, and bacteria. In addition, microbial cell wall preparations and whole cells rich in beta glucans are also suitable sources for beta glucan preparations useful for the present invention. Monomer residues in glucans can have 1-3 and 1-4, or 1-3 and 1-6 linkages (that is the monomer units are joined through 1,3, 1,4 or 1,6 bonds) and the percent of each type can vary. Preferably, beta glucans derived from yeast, particularly from Saccharomyces, preferably Saccharomyces cerevisiae, are used for the present invention. It will be appreciated, however, that other beta glucans would also be suitable. Further examples for suitable biopolymers are chitin and its derivatives, preferably oligoglucosamin and chitosan which represents a typical hydrocolloid.

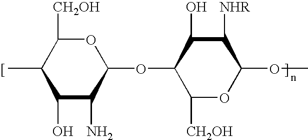

Chitosan is obtained by deacetylisation of chitin and shows molecular weights in the range of 50,000 up to 2,000,000.

### Polyphenols and Plant Extracts Rich in Polyphenols

Suitable non-limiting examples for polyphenols and plant extracts, which are rich in polyphenols, for use in the invention are described hereinafter:

Ginkgo biloba
The active ingredients of the extract are flavonoid glycosides, which among others contain (iso)quercitin glycosides, kaempferol, kaempferol-3-rhamnosides, isorhamnetin, luteoline glycosides, sitosterol glycosides and predominantly hexacyclic terpene lactones, consisting of ginkgolides A, B, C, J, M and bilobalides.

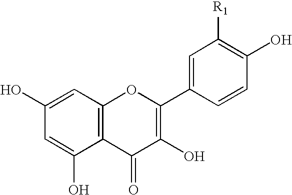

Isorhamnetin (R¹═H), Kaempferol (R¹═OH), Ginkgolid A (R¹═OMe)

Oleacea europensis
The main constituent of the leaves of the olive tree (Oleacea europensis) is the anti-oxidant oleuropein, which is also the main source for hydroxytyrosol.

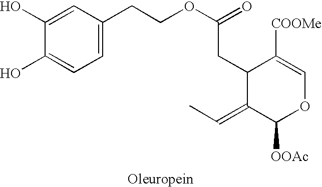

Camellia sinensis
Polyphenols of the catechin and flavonoid type, so-called “tea-tannins” represent the main active principles of extracts of Green Tea (Camellia sinensis):

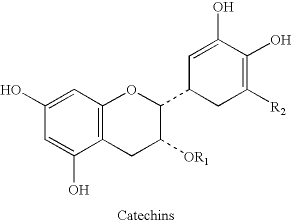

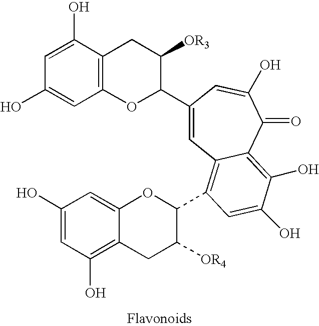

| | R1 | R2 | R3 | R4 |
|---|---|---|---|---|
| (−)-Epicatechin | H | H | | |
| (−) Epigallocatechin | H | OH | | |
| (−) Epicatechin gallate | Galloyl | H | | |
| (−) Epigallocatechin gallate | Galloyl | OH | | |
| Theflavin | | | H | H |
| Theaflavin monogallate A | | | Galloyl | H |
| Theaflavin monogallat B | | | H | Galloyl |
| Theaflavin digallate | | | Galloyl | Galloyl |

Trifolium pratense
The main active principles of red clover (Triflolium pratense) are isoflavones, like e.g. daidzein, genestein, formononentin and biochanin as well as their glucosides like ononin or sissostrin:

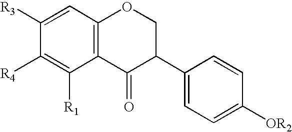

| Isoflavonglucosides | R₁ | R₂ | R₃ | R₄ |
|---|---|---|---|---|
| Daizidin | H | H | Glucose | H |
| Genistin | H | H | Glucose | OH |
| Ononin | H | CH₃ | Glucose | H |
| Sissostrin | H | CH₃ | Glucose | OH |

Litchi Sinensis
Extracts of pericarps from Litchi (Litchi sinensis) are well known for their high content of flavon derivatives like e.g. 2-phenyl-4H-1-benzopyrans, flavanen, flavan-3-ols (catechins, catechin oligomeren), flavan-3,4-diols (leucoanthocyaniden), flavons, flavonols and flavonons. The main component, however, represent condensed tannins, so-called procyanodols (OPC). These compounds comprise 2 to 8 monomers of the catechin or epicatechin-type, like e.g. procyanidins, proanthocynidins, procyanidoel, oligoprocyanidins, leucoanthocyanidins, leucodelphinins, leucocyanins and anthocyanogens. OPC, mainly the preferred proanthocyanidin A2 (OPC A2) behave like vitamin P, especially with respect to MMP inhibition.

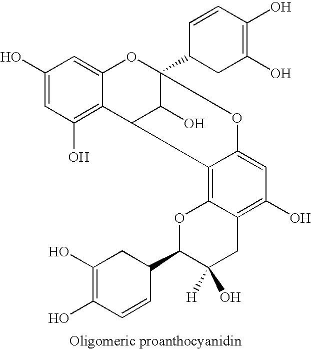

Passiflora incarnata
Extracts of passion flower (Passiflora incarnate) are rich in flavons of the apigenin and luteolin-type and their C-glycosides:

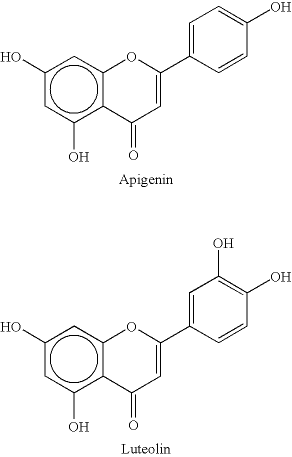

In addition, they comprise 2-B-D-glucosides, schaftosides and iso-schaftosides, isovitexin, isoorientin, vicenin-2, incenin-2, daponanin and trace elements like calcium, phosphor und iron.

Medicago sativa
Extracts of Alfalfa (Medicago sativa) are rich in isoflavons like e.g. daidzein, genestein, formononetin, biochanin A und tricin:

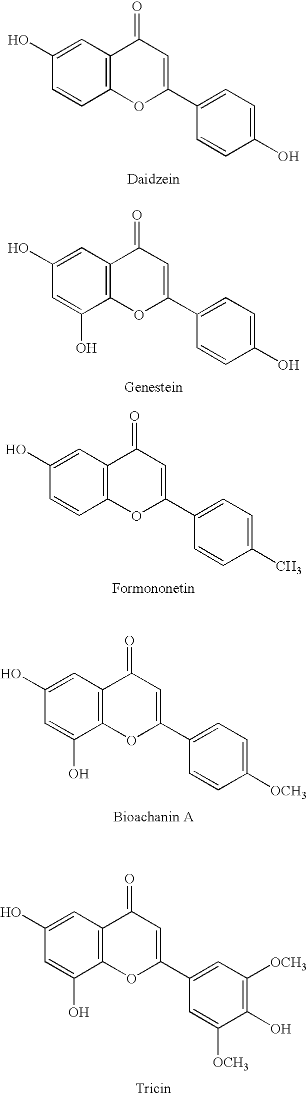

Oral and/or Topical Compositions

The oral and/or topical compositions according to the present invention may comprise the prebiotics and the polyphenols in a weight ratio of 99 to 1 to 50:50 and more particularly 95:10 to 75:25. The highest synergistic effects, however, are observed at ratios of 92:8 to 80:20. In general, the compositions can be used in a concentration of up to about 10, particularly 0.5 to 8 and more particularly 1 to 2% b.w.—calculated on the probiotic micro-organisms being present in the final food composition. One percent, has been found to be particularly suitable.

In another embodiment of the present invention, the compositions are macro- or micro-encapsulated. “Microcapsules” are understood to be spherical aggregates with a diameter of about 0.1 to about 5 mm which contain at least one solid or liquid core surrounded by at least one continuous membrane. More precisely, they are finely dispersed liquid or solid phases coated with film-forming polymers, in the production of which the polymers are deposited onto the material to be encapsulated after emulsification and coacervation or interfacial polymerization. In another process, liquid active materials are absorbed in a matrix (“microsponge”) and, as microparticles, may be additionally coated with film-forming polymers. The microscopically small capsules, also known as nanocapsules, can be dried in the same way as powders. Besides single-core microcapsules, there are also multiple-core aggregates, also known as microspheres, which contain two or more cores distributed in the continuous membrane material. In addition, single-core or multiple-core microcapsules may be surrounded by additional membranes. The membrane may be comprised of natural, semisynthetic or synthetic materials. Natural membrane materials are, for example, gum arabic, agar agar, agarose, maltodextrins, alginic acid and salts thereof, such as sodium or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes. Semisynthetic membrane materials are inter alia chemically modified celluloses, more particularly cellulose esters and ethers, for example cellulose acetate, ethyl cellulose, hydroxypropyl cellulose, hydroxypropyl methyl cellulose and carboxymethyl cellulose, and starch derivatives, more particularly starch ethers and esters. Synthetic membrane materials are, for example, polymers, such as polyacrylates, polyamides, polyvinyl alcohol or polyvinyl pyrrolidone. Examples of known microcapsules are the following commercial products (the membrane material is shown in brackets) Hallcrest Microcapsules (gelatin, gum arabic), Coletica Thalaspheres (maritime collagen), Lipotec Millicapseln (alginic acid, agar agar), Induchem Unispheres (lactose, microcrystalline cellulose, hydroxypropylmethyl cellulose), Unicerin C30 (lactose, microcrystalline cellulose, hydroxypropylmethyl cellulose), Kobo Glycospheres (modified starch, fatty acid esters, phospholipids), Softspheres (modified agar agar), Kuhs Probiol Nanospheres (phospholipids) and Primaspheres or Primasponges (chitosan, anionic polymers). The encapsulation of the compositions according to the present invention is preferred where the active is intended to be liberated at the same part of the intestine. Therefore, one skilled in the art can easily select the adequate encapsulation system by comparing the stability of the capsules under the pH-conditions of the respective part of the intestine.

### Food Compositions

A further embodiment of the present invention relates to food compositions, comprising

(a) prebiotics; and

(b) polyphenols or plant extracts rich in polyphenols, and mixtures thereof.

### INDUSTRIAL APPLICATION

Another embodiment of the present invention is related to the use of mixtures, comprising

(a) prebiotics; and

(b) polyphenols or plant extracts rich in polyphenols, and mixtures thereof,

for stimulating the growth of healthy bacteria, for example in the stomach (if administered orally) or on skin (if applied topically) and for improving the health of the human body, for example with respect to
reduction of Heliobacter pylori infection,
reduction of allergic symptoms,
relief from constipation,
relief from inflammatory bowel syndrome and inflammations of the intestine,
beneficial effects from mineral metabolism, particularly bone density and stability (osteoporosis prevention),
cancer prevention, and
reduction of cholesterol and triacylglycerol plasma concentrations.

The following Examples are illustrative of the invention and should not be considered as limiting the scope of the invention in any manner whatsoever.

### EXAMPLES

### Examples 1 to 10, Comparative Examples C1 to C18

The stimulation of growth of micro-organisms has been studied by enumerating bifidobacterium and lactobacilli in vitro in the presence of various test substances:

Extract A: Extract of Trifolium pratense (Red clover)
Extract B: Extract of Camellia sinensis (Green tea)
Extract C: Extract of Oleacea europensis (Olive tree)
Extract D: Extract of Ginkgo biloba (Ginkgo tree)

All extracts possess an active content of about 20% b.w. and are commercially available from Cognis Deutschland GmbH & Co. KG.

Aliquots (1 mL) of human fecal homogenates (10 g per 100 mL diluent) were added to diluted WC broth (diluted 50:50 with 0.05M phosphate buffer) to which were added the test mixtures and a lactobacillus or bifidobacterium strain. For each of the combinations, parallel tubes were prepared with one set being inoculated with Bifidobacterium spp or Lactobacillus spp. All mixtures were then incubated for up to 24 hours and bacterial numbers determined. The results are presented in Tables 1 and 2 (amount of extract calculated on active content):

TABLE 1
Effect of 1% prebiotic, plant extracts and prebiotic/plant extract mixture on Bifidobacterium

| | 0 | C1 | C2 | C3 | C4 | C5 | C6 | C7 | C8 | 1 | 2 | 3 | 4 | 5 |
|---|---|---|---|---|---|---|---|---|---|---|---|---|---|---|
| Inulin | — | 1.0 | — | — | — | — | — | — | — | 0.8 | 0.9 | — | — | — |
| Lactosucrose | — | — | 1.0 | — | — | — | — | — | — | — | — | 0.9 | — | — |
| Lactolin | — | — | — | 1.0 | — | — | — | — | — | — | — | — | 0.9 | — |
| Betaglucan | — | — | — | — | 1.0 | — | — | — | — | — | — | — | — | 0.9 |
| Extract A | — | — | — | — | — | 1.0 | — | — | — | 0.2 | — | — | — | — |
| Extract B | — | — | — | — | — | — | 1.0 | — | — | — | 0.1 | 0.1 | — | — |
| Extract C | — | — | — | — | — | — | — | 1.0 | — | — | — | — | 0.1 | — |
| Extract D | — | — | — | — | — | — | — | — | 1.0 | — | — | — | — | 0.1 |
| Bacterial | 1.0 × | 1.5 × | 1.1 × | 1.6 × | 1.2 × | 3.3 × | 2.3 × | 2.7 × | 3.4 × | 4.1 × | 4.3 × | 4.0 × | 4.0 × | 4.4 × |
| numbers | 10⁶ | 10⁷ | 10⁷ | 10⁷ | 10⁷ | 10⁶ | 10⁶ | 10⁶ | 10⁶ | 10⁷ | 10⁷ | 10⁷ | 10⁷ | 10⁷ |
| (CFU/ml) | | | | | | | | | | | | | | |

Starting from a control of 1.0×10⁶ CFU/ml (O), the addition of 1% b.w. of various prebiotics (Comparative Examples C1-C4) increases the CFU by a factor of 10, while the addition of the plant extracts had only a weak effect on the stimulation of cell growth (Comparative Examples C5-C8). Adding, however, mixture of prebiotics and plant extracts to the samples, the CFU numbers were multiplied by a factor of about 40 (Inventive Examples 1 to 5). The highest synergistic effect can be seen at a ratio prebiotic: polyphenols of about 90:10.

TABLE 2
Effect of 1% prebiotic, plant extract and prebiotic/plaut extract mixture on Lactobacterium

| | 0 | C9 | C10 | C11 | C12 | C13 | C14 | C15 | C16 | 6 | 7 | 8 | 9 | 10 |
|---|---|---|---|---|---|---|---|---|---|---|---|---|---|---|
| Inulin | — | 1.0 | — | — | — | — | — | — | — | 0.8 | 0.9 | — | — | — |
| Lactosucrose | — | — | 1.0 | — | — | — | — | — | — | — | — | 0.9 | — | — |
| Lactolin | — | — | — | 1.0 | — | — | — | — | — | — | — | — | 0.9 | — |
| Betaglucan | — | — | — | — | 1.0 | — | — | — | — | — | — | — | — | 0.9 |
| Extract A | — | — | — | — | — | 1.0 | — | — | — | 0.2 | — | — | — | — |
| Extract B | — | — | — | — | — | — | 1.0 | — | — | — | 0.1 | 0.1 | — | — |
| Extract C | — | — | — | — | — | — | — | 1.0 | — | — | — | — | 0.1 | — |
| Extract D | — | — | — | — | — | — | — | — | 1.0 | — | — | — | — | 0.1 |
| Bacterial | 2.8 × | 1.4 × | 1.1 × | 1.5 × | 1.1 × | 4.2 × | 4.3 × | 4.4 × | 4.5 × | 6.5 × | 6.3 × | 6.7 × | 6.6 × | 6.9 × |
| numbers | 10⁵ | 10⁶ | 10⁶ | 10⁶ | 10⁶ | 10⁵ | 10⁵ | 10⁵ | 10⁵ | 10⁵ | 10⁶ | 10⁶ | 10⁶ | 10⁶ |
| CFU/ml) | | | | | | | | | | | | | | |

Starting from a control of 2.8×10⁵ CFU/ml (O), the addition of 1% b.w. of various prebiotics (Comparative Examples C9-C12) increases the CFU by a factor of 4, while the addition of the plant extracts had only a weak effect on the stimulation of cell growth (Comparative Examples C13-C16). Adding however, mixture of prebiotics and plant extracts to the samples, the CFU numbers were multiplied by a factor of about 15 (Inventive Examples 6 to 10). The highest synergistic effect was observed at a ratio prebiotic:polyphenols of about 90:10.

### Example 11

### Yogurt Composition

Soy milk is added to 15-75 parts by volume of cow milk to make 100 parts of the mixture. The mixture is then pasteurised at about 90° C. for 15 seconds and then cooled. The cooled, pasteurised mixtures are then inoculated with 3 to 5 percent by volume of a yogurt culture having 1:1 ratio of Lactobacillus bulgaricus and Bifidobacterium adolescentis. The incubation is carried out at about 42° C. In about 2 hours, thickening will occur. The fermentation is carried out for about 5.5 hours. The yogurt compositions thus obtained is treated with 1%—calculated on the amount of micro-organisms being present—of a 9:1 mixture of inulin and an extract of Green Tea. The products have a firm consistency and a flavor like or substantially indistinguishable from that of a corresponding yogurt composition using 100 percent of fresh cow milk. A small amount of citric acid can be added to the fermentation mixture to enhance the flavor of the final yogurt composition. A suitable amount of citric acid is 0.5 percent based on the weight of the composition.

What we claim is:

1. A composition for topical and/or oral administration, comprising
(a) a prebiotic; and
(b) a polyphenol or a plant extract containing a polyphenol, and mixtures thereof.

2. A composition according to claim 1, wherein said prebiotic (component a) is selected from the group consisting of a fructooligosaccharide, an inulin, an isomaltooligosaccharide, a lactilol, a lactosucrose, a lactulose, a pyrodextrin, a soy oligosaccharide, a transgalactooligosaccharide, a xylooligosaccharide, and a biopolymer, and mixtures thereof.

3. A composition according to claim 1, wherein said polyphenol (component b) is selected from the group consisting of an (iso)flavon, an (iso)flavonol, an (iso)flavonon, an (iso)flavonoid, a catechin, a ginkgolide selected from A, B, and C, a bilobalide, an oliogoprocyanidin, and its glycosides, and mixtures thereof.

4. A composition according to claim 1 wherein said polyphenol is selected from the group consisting of (iso)quercitin, kaempferol, isorhamnetin, luteolin, oleuropein, hydroxytyrosol, (epi)catechin(gallate), (epi)gallocatechin(gallate), theaflavin(gallate), daidzein, genestein, formononentin, biochanin A, tricin, proanthocyanidin A2, apigenin luteolin and their glycosides, and mixtures thereof.

5. A composition according to claim 1 wherein component (b) is an extract selected from the group consisting of Ginkgo biloba, Camellia sinensis, Trifolium pratense, Oleacea europensis, Litchi sinensis, Passiflora incarnata, Medicago sativa, and mixtures thereof.

6. A composition according to claim 1 wherein components (a) and (b) are present in weight ratios ranging from 99:1 to 50:50.

7. A composition according to claim 1 wherein components (a) and (b) are present in an amount of up to 10% b.w., based on the presence of a microorganism in the composition.

8. A composition according to claim 1 wherein said components (a) and (b) are macro- or micro-encapsulated.

9. The composition according to claim 1 wherein the composition is administered orally.

10. The composition according to claim 1 wherein the composition is applied topically.
